(19) United States
(12) Patent Application Publication
(10) Pub. No.: US 2020/0108217 A1
(43) Pub. Date: Apr. 9, 2020

(54) TRACHEOSTOMY TUBE HOLDER ATTACHMENT

(71) Applicant: Cynthia Vickers, Warner Robins, GA (US)

(72) Inventor: Cynthia Vickers, Warner Robins, GA (US)

(21) Appl. No.: 16/594,323

(22) Filed: Oct. 7, 2019

Related U.S. Application Data
(60) Provisional application No. 62/741,731, filed on Oct. 5, 2018.

(51) Int. Cl. A61M 16/04; A61M 25/02

(52) U.S. Cl. CPC A61M 16/0465; A61M 2025/026; A61M 2025/0213; A61M 25/02

(57) ABSTRACT

An exemplary embodiment provides a tracheal tube system, comprising a tracheostomy tube holder and a cover. The tracheostomy tube holder for affixing a tracheal tube to a patient, the tracheostomy tube holder comprising a first end, a second end, and a connector for attaching the first end to the second end. The cover configured to surround at least a portion of the connector to thereby inhibit access to the connector.

### CROSS-REFERENCE TO RELATED APPLICATIONS

This application claims the benefit of U.S. Provisional Application Ser. No. 62/741,731, filed on Oct. 4, 2018, which is incorporated herein by reference in its entirety as if fully set forth below.

### TECHNICAL FIELD OF THE INVENTION

The various embodiments of the present disclosure relate generally to relate to covers and, more particularly, to systems and methods for covering a tracheostomy tube holder to decrease the likelihood of mistaken dislodgement.

### BACKGROUND OF THE INVENTION

Tracheostomy tube holders are medical devices that provide a receptacle through which a tracheal tube can be inserted, providing stability to the tracheal tube, in order to, for example, establish an airway for a patient that requires an artificial airway. Tracheostomy tube holders therefore serve a critical function for patients needing an artificial airway by, for example, providing a stable position for a tracheal tube to interact with the patient, often by aligning the tracheal tube with a fistula in the patient's neck.

Conventional tracheal tube holders are designed to align the tracheal tube with the patient's mouth or through a fistula in the neck. These designs are fastened to the patient by various means, including necklaces and Velcro or hook-and-loop fasteners. Additionally, these designs provide a wide range of size and a wide range of materials that interact with the skin of the patient. Many of these sizes and materials induce anxiety or make the user uncomfortable. For example, if the design uses a plastic that makes contact with the skin of the patient, the patient is often made uncomfortable by the abrasion of the plastic against the patient's skin. Additionally, for example, designs incorporating a large or wide strap or other component can make the patient uncomfortable.

As shown in FIG. 1, an example of a conventional tracheostomy tube holder 100 is shown. The tracheostomy tube holder 100 comprises an orifice 105 through which or into which the tracheal tube is inserted to interact with the patient. Further, the tracheostomy tube holder 100 comprises a necklace 110 made from a thin flexible material. The necklace 110 wraps around the patient's neck and is secured to itself via a hook-and-loop fastener 115. The hook-and-loop fastener 115 can be readily engaged and disengaged by the patient. The ability to readily engage and disengage the hook-and-loop fastener 115 can be a disadvantage or even dangerous to the patient. This is because the patient may not understand that the tracheostomy tube holder supports a tracheal tube that is providing them with oxygen, for example, and that removing the tracheostomy tube holder could cause them to be seriously injured or die. This may be especially true if the patient is a young child or somehow mentally impaired.

Accordingly, there is a need for a tracheostomy tube holder or another device that reduces the ability of the patient to remove the tracheostomy tube holder. More specifically, there is a need for a cover that makes it more difficult for a patient, especially a child, to remove their tracheostomy tube holder. Various embodiments of the present invention address these desires.

### BRIEF SUMMARY OF THE INVENTION

Briefly described, embodiments of the present invention relate to a cover for a tracheostomy tube holder or an enhanced tracheostomy tube holder. The cover can be configured in a way as to inhibit a patient, especially a child, from easily removing a tracheal tube. In some embodiments, the cover comprises snaps that allow a cloth or piece of fabric to be secured to itself, around the necklace of a tracheostomy tube holder, via a male and female snap forming an interference fit. The secured cover creating a barrier that prevents a patient from readily dislodging their tracheostomy tube holder by, for example, disengaging the hook-and-loop fastener securing the necklace of the tracheostomy tube holder. The tracheostomy tube holder is therefore more difficultly removed and has a reduced likelihood of being mistakenly removed by a patient.

In some embodiments, the enhanced tracheostomy tube holder comprises a cover that is not easily removable from the enhanced tracheostomy tube holder. For example, the cover could be secured to the enhanced tracheostomy tube holder. This enhanced tracheostomy tube holder could therefore use an integrated cover to create a barrier that prevents a patient from readily dislodging their tracheostomy tube holder. The tracheostomy tube holder is therefore more difficult to remove and has a reduced likelihood of being mistakenly removed by a patient.

Another aspect of the present disclosure may involve a method for assembling a cover or an enhanced tracheostomy tube holder. In some embodiments, a method may comprise providing a single sheet of a flexible material. A method of the present disclosure may further comprise joining a first portion of the flexible material to a second portion of the flexible material with an interference snap, and joining the flexible material so that it substantially covers the hook-and-loop fasteners used to secure a necklace of a tracheostomy tube holder.

According to the present disclosure, two or more portions of the flexible material could be joined by interference fit snaps, sewing, laminating, gluing, adhering, fastening, attaching, buttoning, zipping, or sticking a first portion of the flexible material to a second portion of the flexible material. Furthermore, a method according to the present disclosure may comprise joining the flexible material or cover with a tracheostomy tube holder to create an enhanced tracheostomy tube holder. The flexible material can be joined with the tracheostomy tube holder by, for example, interference fit snaps, sewing, laminating, gluing, adhering, fastening, attaching, buttoning, zipping, or sticking a first portion of the flexible material to a second portion of the flexible material

Some embodiments further relate to tracheal tube systems and methods of attaching a tracheal tube to a patient. An exemplary embodiment provides a tracheal tube system, comprising a tracheostomy tube holder and a cover. The tracheostomy tube holder can be for affixing a tracheal tube to a patient. The tracheostomy tube holder comprising a first end, a second end, and a connector for attaching the first end to the second end. The cover configured to surround at least a portion of the connector to thereby inhibit access to the connector.

In any of the embodiments disclosed herein, the cover can comprise a first side, a second side, and at least one fastener configured to attach the first side to the second side.

In any of the embodiments disclosed herein, the at least one fastener can comprise a plurality of snaps.

In any of the embodiments disclosed herein, the at least one fastener can comprise a plurality of buttons.

In any of the embodiments disclosed herein, the at least one fastener can be positioned along at least one of the first or second sides of the cover.

In any of the embodiments disclosed herein, the cover comprises fabric.

In any of the embodiments disclosed herein, the cover can have a length greater than a length of the connector.

In any of the embodiments disclosed herein, the system can further comprise a tracheal tube attached to the tracheostomy tube holder.

Another embodiment provides a tracheal tube system comprising a tracheal tube, a first strap, a second strap, a connector, and a cover. The first strap can have a first end attached to the tracheal tube and a second end. The second strap can have a first end attached to the tracheal tube and a second end. The connector can be configured to attach the second end of the first strap to the second end of the second strap. The cover can be configured to at least partially enclose the connector.

Another embodiment provides a method of attaching a tracheal tube to a patient comprising: providing a tracheostomy tube holder attached to a tracheal tube, the tracheostomy tube holder comprising a first end, a second end, and a connector for attaching the first end to the second end; attaching the first end to the second end with the connector; and placing a cover over at least a portion of the connector.

In any of the embodiments disclosed herein, the cover can comprise a first side, a second side, and at least one fastener for attaching the first side to the second side. Placing the cover over at least a portion of the connector can comprise: placing the cover adjacent the connector; folding at least one of the first side and the second side of the cover over a portion of the connector; and attaching the first side of the cover to the second side of the cover using the at least one fastener, such that the cover defines an interior cavity, the interior cavity containing at least a portion of the connector.

In any of the embodiments disclosed herein, the interior cavity can have a length substantially equal to a length of the cover.

In any of the embodiments disclosed herein, placing the cover over at least a portion of the connector can result in the entire connector being surrounded by the cover.

In any of the embodiments disclosed herein, at least one fastener comprises a plurality of snaps.

In any of the embodiments disclosed herein, the cover can comprise a first end, a second end, and an internal cavity between the first end and the second end, the internal cavity containing at least a portion of the connector.

These and other aspects of the present invention are described in the Detailed Description of the Invention below and the accompanying figures. Other aspects and features of embodiments of the present invention will become apparent to those of ordinary skill in the art upon reviewing the following description of specific, exemplary embodiments of the present invention in concert with the figures. While features of the present invention may be discussed relative to certain embodiments and figures, all embodiments of the present invention can include one or more of the features discussed herein. Further, while one or more embodiments may be discussed as having certain advantageous features, one or more of such features may also be used with the various embodiments of the invention discussed herein. In similar fashion, while exemplary embodiments may be discussed below as device, system, or method embodiments, it is to be understood that such exemplary embodiments can be implemented in various devices, systems, and methods of the present invention.

### BRIEF DESCRIPTION OF THE DRAWINGS

The following Detailed Description of the Invention is better understood when read in conjunction with the appended drawings. For the purposes of illustration, there is shown in the drawings exemplary embodiments, but the subject matter is not limited to the specific elements and instrumentalities disclosed.

FIG. 1 is a perspective view of a conventional tracheostomy tube holder, showing the tracheostomy tube holder with its hook-and-loop fastener secured to itself.

FIG. 2 is a perspective view of a conventional tracheostomy tube holder, showing the tracheostomy tube holder with its hook-and-loop fastener unsecured.

FIG. 3 is a perspective view of a cover for a tracheostomy tube holder, in accordance with some embodiments of the present invention.

FIG. 4 is a second perspective view of a cover for a tracheostomy tube holder, in accordance with some embodiments of the present invention.

FIG. 5 is a perspective view of a cover for a tracheostomy tube holder covering a tracheostomy tube holder, in accordance with some embodiments of the present invention.

FIG. 6 is a second perspective view of a cover for a tracheostomy tube holder covering a tracheostomy tube holder, in accordance with some embodiments of the present invention.

### DETAILED DESCRIPTION OF THE INVENTION

To facilitate an understanding of the principles and features of the various embodiments of the invention, various illustrative embodiments are explained below. Although certain embodiments of the invention are explained in detail as being a cover or an enhanced tracheostomy tube holder, it is to be understood that other embodiments are contemplated, such as embodiments of enhanced tracheostomy tube holders with more sophisticated means of securing the necklace of the tracheostomy tube holder. Accordingly, where the terms “cover”, “enhanced tracheostomy tube holder”, and related terms are used throughout this disclosure, it will be understood that other entities, objects, or activities can take the place of these in various embodiments of the invention. For example, and not limitation, some embodiments of the invention may improve the necklace of a tracheostomy tube holder. It is not intended that the invention is limited in its scope to the details of construction and arrangement of components set forth in the following description or examples. The invention is capable of other embodiments and of being practiced or carried out in various ways. Also, in describing the embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. Furthermore, it is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

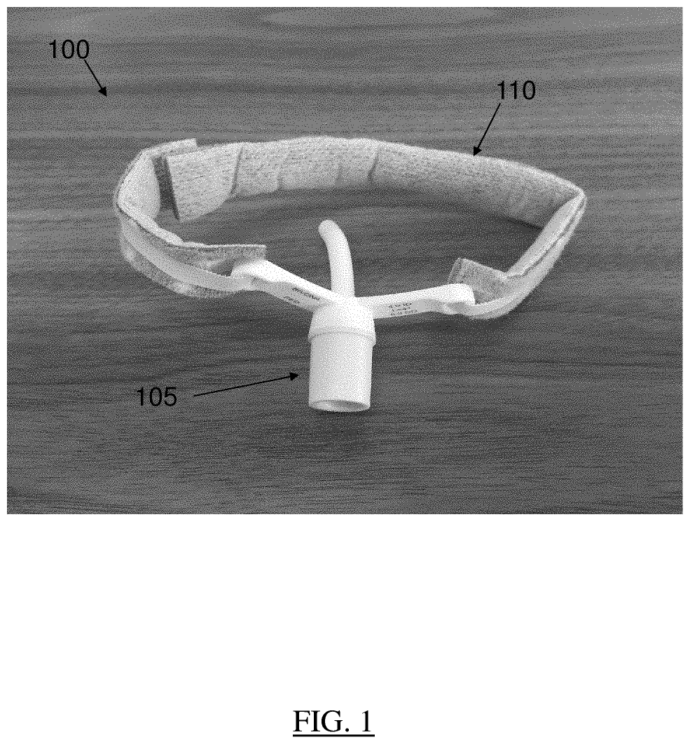

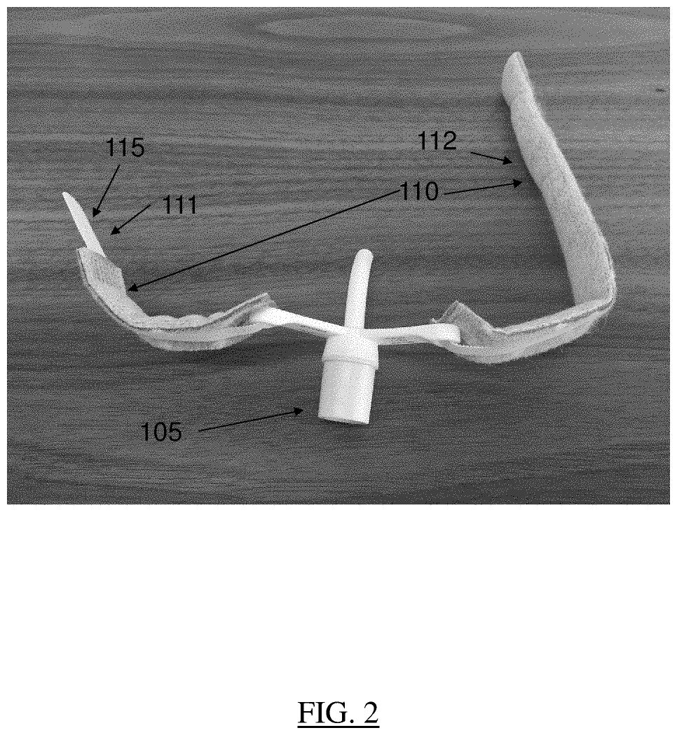

As explained above, and as shown in FIGS. 1-2, a problem with traditional tracheostomy tube holders 100 is that they comprise a necklace 110, which can comprise a first strap 111 and a second strap 112, that can be easily disengaged, which can be dangerous or even life threating to the patient because a tracheostomy tube holder 100 is commonly used to align a tracheal tube providing oxygen to the patient. Misalignment of the tracheal tube can prevent oxygen from reaching the patient. The tracheostomy tube holder 100 is easily misaligned or removed because it is often secured via a readily disengaged necklace 110. For example, a necklace 110 using a hook-and-loop fastener 115 (as a connector) is readily accessed by the patient or a third party, and also is readily disengaged from securing the necklace 110, and therefore the tracheostomy tube holder 100, to the patient, by either the patient or a third party. As outlined above, this can cause serious harm and even death if the tracheostomy tube holder 100 is being used in conjunction with a tracheal tube to provide the patient with medical treatment.

It would therefore be beneficial to minimize or eliminate the ability to misalign the tracheostomy tube holder 100 by disengaging its necklace 110. Embodiments of the present invention provide several solutions to this problem. In some embodiments, the present invention can comprise a cover for a necklace. Similarly, embodiments of the present invention can include an enhanced tracheostomy tube holder that comprises a cover incorporated into the necklace of the tracheostomy tube holder. Accordingly, some embodiments of the present invention can minimize or eliminate the potential that the tracheostomy tube holder can be misaligned or removed when its necklace is mistakenly disengaged.

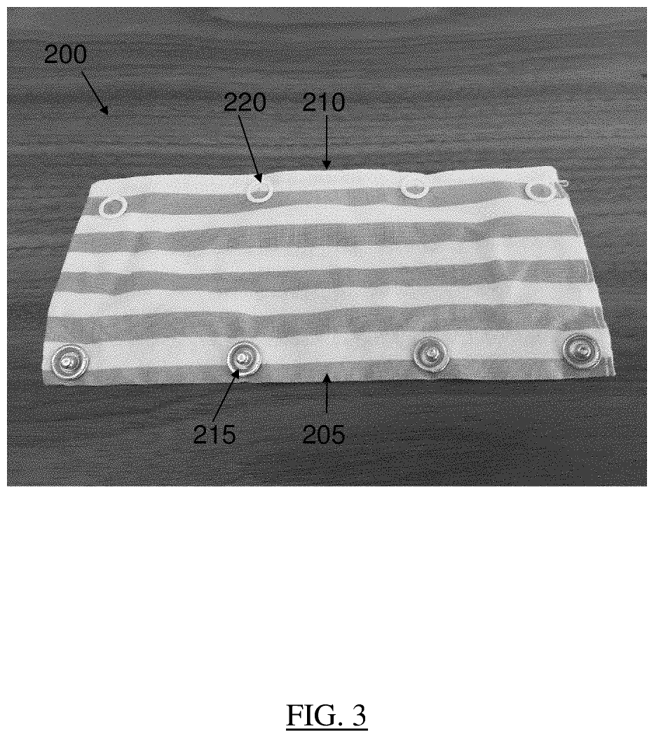

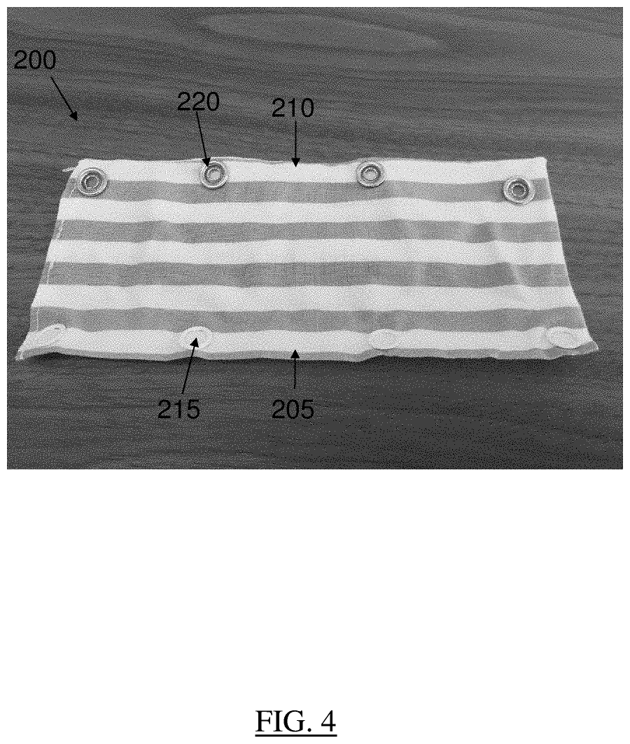

As shown in FIGS. 3 and 4, embodiments of the present disclosure can be a cover 200. The cover 200 can be a piece of fabric, such as cotton, and can comprise fasteners for joining a first side of the cover 205 to a second side of the cover 210. For example, a first side of the cover can be joined to a second side of the cover by friction fit snaps, which can comprise a male end 215 and a female end 220. The male end 215 can be inserted into the female end 220 to form a friction fit. When the friction fit snaps are joined, the cover 200 can form an internal cavity, e.g., a tube or cylinder, through which a necklace of a tracheostomy tube holder can run. The cover 200 can thereby shield the necklace or the connector 115 of the necklace, for example, from a patient or necklace wearer, making it more difficult for the patient or necklace wearer to remove or misalign, for example, the necklace or a tracheostomy tube holder by, for example, disengaging the hook-and-loop connection on the tracheostomy tube holder necklace. In other words, the cover can surround the connector 115 or at least a portion of the connector 115, thus inhibiting the ability to access the connector and remove or displace the tracheal tube.

Although FIGS. 3-4 illustrate the cover including fasteners in the form of snaps, many other fasteners are contemplated. For example, in some embodiments, the fasteners can include buttons. Such buttons may comprise a button on a first side 205 of the cover and a slit through which the button could be inserted on a second side 210 of the cover. Similarly, the fastener could include Velcro on each of the first and second sides 205 210 of the cover for attaching the first side 205 to the second side 210. Additionally, although FIGS. 3-4 illustrate a cover comprising four fasteners, the invention is not so limited. Rather, various embodiments can include different numbers of fasteners, e.g., one, two, three, four, five, etc.

In some embodiments, the cover can be tube shaped, i.e., having a first end and a second end, and a hollow cavity therebetween. Such an embodiment may not include fasteners as the tube shape does not require a first side to be attached to a second side to create an internal cavity. In such an embodiment, during assembly, a first strap 111 of the holder can be inserted into the cavity and through both ends of the cover. The first strap 111 can then be connected to the second strap 112. The cover than then be slid back over the connector 115 of the holder to inhibit the holder from being disconnected.

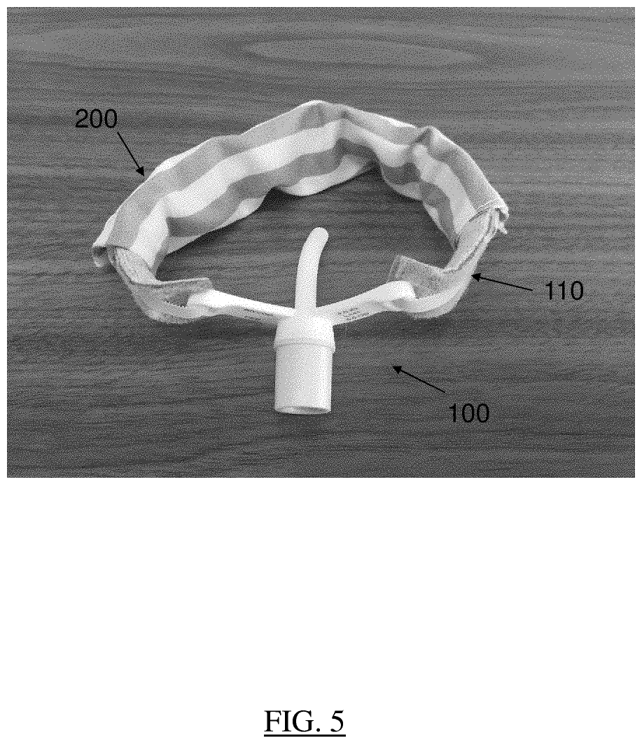

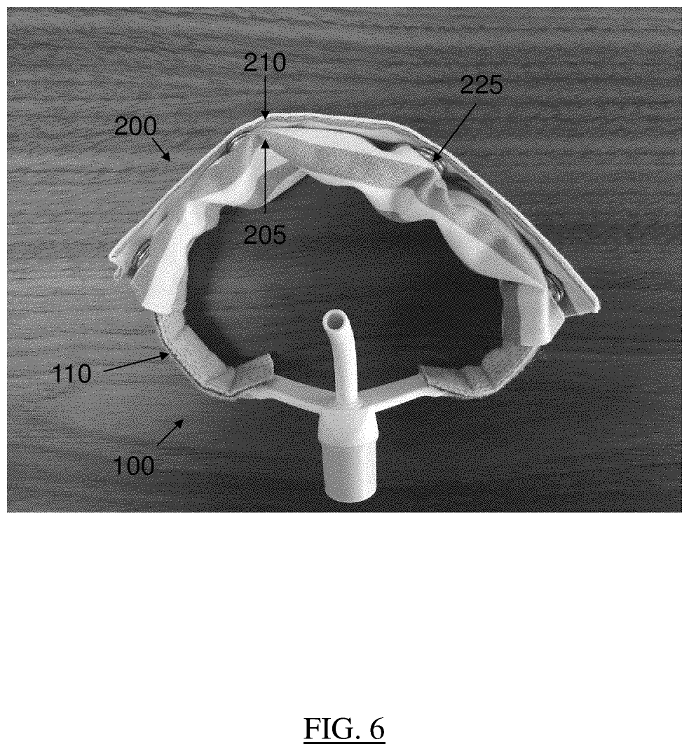

Attention is turned to FIG. 5, which shows an example embodiment of the present disclosure where a cover 200 is covering a portion necklace 110 of a tracheostomy tube holder 100. The tracheostomy tube holder necklace, particularly the connector 115, is therefore not as easily accessed, and the hook-and-loop connection 115 on the necklace of a tracheostomy tube holder is not as readily disengaged. Further, attention is turned to FIG. 6, which shows an example embodiment of the present disclosure and further shows how a first portion of the cover 205 can be joined to a second portion of the cover 210 by joined friction fit snaps 225.

The cover 200 can be made from one or more pieces of material. The cover 200 can additionally be any suitable piece of material or fabric. For example, the cover may comprise cotton, silk, polyester, velvet, wool, denim, satin, damask, canvas, linen crepe, corduroy, shantung, rayon, sateen, or other types of fabrics, both natural and synthetic. The cover may comprise more than one type of fabric, and may contain both natural and synthetic fabrics or fibers.

Generally, it may be beneficial for the cover 200 to be made from a fabric or material that is comfortable for the patient, as it will generally be in contact with the patient's skin and/or neck area. For example, the cover may comprise the natural and/or synthetic fibers listed above. In some embodiments, however, it may be beneficial for the cover to comprise materials that are more readily sterilized. For example, if the patient is in a hospital setting or if the patient has a weakened immune system. In some embodiments, therefore, the cover 200 may comprise a plastic or other synthetic material that is readily sterilized or disinfected, including, but not limited to, rubber, silicone, latex, plastic, and combinations thereof. A person of skill in the art will understand which types of materials are best given the needs of the patient.

Embodiments of the present disclosure comprise covers for tracheostomy tube holders and enhanced tracheostomy tube holders, but also include covers for other types of medical instruments and fixtures that there exists a desire to prevent ready access to the means of misaligning or removing the instrument or fixture. For example, a cover could be used to prevent removal of medical device bracelet worn on the wrist. A person of skill in the art will understand that the cover systems and methods described herein are therefore adaptable and will use them given the needs of the patient or user.

The cover 200 can comprise various means for joining a first portion of the cover 205 to a second portion of the cover 210. For example, friction fit snaps, sewing, laminating, gluing, adhering, fastening, attaching, buttoning, zipping, sticking, or hook-and-loop fasteners, and combinations thereof. Additionally, embodiments of the present disclosure may comprise more than one means for joining a first portion of the cover 205 to a second portion of the cover 210. For example, the cover may use both friction fit snaps and zippers. This may serve to further minimize the ability of the patient to mistakenly misalign or remove the tracheostomy tube holder. Further, embodiments of the present invention can use alternating means of joining a first portion of the cover 205 to a second portion of the cover 210. For example, alternating the use of friction fit snaps and buttons. As an additional example, buttons could be used but alternated so that their male and female elements are alternated from the first portion of the cover 205 to the second portion of the cover 210. These alternating means and other various combinations of means for joining a first portion of the cover 205 to a second portion of the cover 210 can serve to make misalignment and/or removal of the tracheostomy tube holder more difficult by, for example, making it more difficult for a less dexterous person, for example a child, to disengage the necklace of the tracheostomy tube holder.

Another aspect of the present disclosure may involve a method for assembling a tracheostomy tube holder cover or an enhanced tracheostomy tube holder. In some embodiments, a method may comprise providing a flexible material with a plurality of means for joining a first portion of the flexible material to a second portion of the flexible material. Additional embodiments may comprise joining a first portion of a flexible material with a second portion of a flexible material with a plurality of means for joining a first portion of a flexible material with a second flexible material, wherein the joined flexible material covers or surrounds a second piece of material. For example, the joined flexible material could cover a tracheostomy tube holder necklace. By covering a tracheostomy tube holder necklace the joined flexible material could, for example, make it more difficult to remove the tracheostomy tube holder. This could happen, for example, by making it more difficult to access the means by which the tracheostomy tube holder necklace is joined to itself. As an additional example, a flexible material could be provided with a plurality of means for joining a first portion of the flexible material to a second portion of the flexible material, such as friction fit snaps, the first portion of the flexible material could be joined to the second portion of the flexible material using the means for joining the two, such as joining using friction fit snaps, and the joining of the flexible material could partially encapsulate a second material, such as a necklace of a tracheostomy tube holder. In some embodiments, a method for using a cover over a tracheostomy tube holder necklace would require someone to dislodge joined friction fit snaps before disengaging hook-and-loop fasteners that are joining a first portion of a tracheostomy tube holder necklace to a second portion of a tracheostomy tube holder necklace.

Some embodiments of the present disclosure may comprise joining a flexible material to a tracheostomy tube holder in a permanent or semi-permanent fashion, such that the flexible material and the tracheostomy tube holder are effectively one component. In this way the tracheostomy tube holder would become an enhanced tracheostomy tube holder. The enhanced tracheostomy tube holder could be used by a method comprising using the flexible material attachment to cover the necklace of the tracheostomy tube holder. In this way, the flexible material could reduce the likelihood that the tracheostomy tube holder would be inadvertently dislodged or removed

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. Instead, it is intended that the invention is defined by the claims appended hereto.

What is claimed is:

1. A tracheal tube system, comprising:
a tracheostomy tube holder for affixing a tracheal tube to a patient, the tracheostomy tube holder comprising a first end, a second end, and a connector for attaching the first end to the second end; and
a cover configured to surround at least a portion of the connector to thereby inhibit access to the connector.

2. The tracheal tube system of claim 1, wherein the cover comprises:
a first side;
a second side; and
at least one fastener configured to attach the first side to the second side.

3. The tracheal tube system of claim 2, wherein the at least one fastener comprises a plurality of snaps.

4. The tracheal tube system of claim 2, wherein the at least one fastener comprises a plurality of buttons.

5. The tracheal tube system of claim 2, wherein the at least one fastener is positioned along at least one of the first or second sides of the cover.

6. The tracheal tube system of claim 1, wherein the cover comprises fabric.

7. The tracheal tube system of claim 1, wherein the cover has a length greater than a length of the connector.

8. The tracheal tube system of claim 1, further comprising a tracheal tube attached to the tracheostomy tube holder.

9. A tracheal tube system, comprising:
a tracheal tube;
a first strap having a first end attached to the tracheal tube and a second end;
a second strap having a first end attached to the tracheal tube and a second end;
a connector configured to attach the second end of the first strap to the second end of the second strap; and
a cover configured to at least partially enclose the connector.

10. The tracheal tube system of claim 9, wherein the cover comprises:
a first side;
a second side; and
at least one fastener configured to attach the first side to the second side.

11. The tracheal tube system of claim 10, wherein the at least one fastener comprises a plurality of snaps.

12. The tracheal tube system of claim 10, wherein the at least one fastener comprises a plurality of buttons.

13. The tracheal tube system of claim 10, wherein the at least one fastener is positioned along at least one of the first or second sides of the cover.

14. The tracheal tube system of claim 9, wherein the cover has a length greater than a length of the connector.

15. A method of attaching a tracheal tube to a patient, comprising:
providing a tracheostomy tube holder attached to a tracheal tube, the tracheostomy tube holder comprising a first end, a second end, and a connector for attaching the first end to the second end;
attaching the first end to the second end with the connector; and
placing a cover over at least a portion of the connector.

16. The method of claim 15, wherein the cover comprises a first side, a second side, and at least one fastener for attaching the first side to the second side, wherein placing the cover over at least a portion of the connector comprises:
placing the cover adjacent the connector;
folding at least one of the first side and the second side of the cover over a portion of the connector; and
attaching the first side of the cover to the second side of the cover using the at least one fastener, such that the cover defines an interior cavity, the interior cavity containing at least a portion of the connector.

17. The method of claim 16, wherein the interior cavity has a length substantially equal to a length of the cover.

18. The method of claim 15, wherein placing the cover over at least a portion of the connector results in the entire connector being surrounded by the cover.

19. The method of claim 16, wherein the at least one fastener comprises a plurality of snaps.

20. The method of claim 15, wherein the cover comprises a first end, a second end, and an internal cavity between the first end and the second end, the internal cavity containing at least a portion of the connector.
